(19) United States
(12) Patent Application Publication
(10) Pub. No.: US 2018/0296080 A1
(43) Pub. Date: Oct. 18, 2018

(54) ADAPTIVE TUNING OF 3D ACQUISITION SPEED FOR DENTAL SURFACE IMAGING

(71) Applicant: Carestream Dental Technology Topco Limited, London (GB)

(72) Inventors: Yannick Glinec, Montevrain (FR); Yanbin Lu, Shanghai (CN)

(21) Appl. No.: 15/766,825

(22) PCT Filed: Nov. 5, 2015
(86) PCT No.: PCT/US2015/059125

§ 371 Date: Apr. 8, 2018

Related U.S. Application Data
(60) Provisional application No. 62/238,753, filed on Oct. 8, 2015.

(51) Int. Cl. A61B 1/24; A61B 1/045; A61C 9/00; A61B 1/00; A61B 1/06; G01B 11/25

(52) U.S. Cl. CPC A61B 1/24; A61B 1/045; A61C 9/006; G01B 2210/52; A61B 1/00009; A61B 1/0661; G01B 11/2518; A61B 1/00006

(57) ABSTRACT

A method for obtaining one or more 3D surface images of a tooth, the method executed at least in part by a computer repeats a sequence of acquiring a succession of images of the tooth from a scanner, in particular an intra-oral structured-light scanner, at a scanner acquisition rate and changing the scanner acquisition rate according to differences between successive images in the acquired succession of images. In particular, said differences are used to determine the relative speed of movement of the scanner and to use this information to adjust the acquisition frequency such that the amount of redundant information in the image data is reduced.

### TECHNICAL FIELD

The disclosure relates generally to the field of diagnostic imaging using structured light and more particularly relates to a method for managing automatic capture of structured light images for three-dimensional imaging of the surface of teeth and other structures.

### BACKGROUND

A number of techniques have been developed for obtaining surface contour information from various types of objects in medical, industrial, and other applications. These techniques include optical 3-dimensional (3-D) measurement methods that provide shape and depth information using images obtained from patterns of light directed onto a surface.

Structured light imaging is one familiar technique that has been successfully applied for surface characterization. In structured light imaging, a pattern of illumination is projected toward the surface of an object from a given angle. The pattern can use parallel lines of light or more complex periodic features, such as sinusoidal lines, dots, or repeated symbols, and the like. The light pattern can be generated in a number of ways, such as using a mask, an arrangement of slits, interferometric methods, or a spatial light modulator, such as a Digital Light Processor from Texas Instruments Inc., Dallas, Tex. or similar digital micromirror device. Multiple patterns of light may be used to provide a type of encoding that helps to increase robustness of pattern detection, particularly in the presence of noise. Light reflected or scattered from the surface is then viewed from another angle as a contour image, taking advantage of triangulation in order to analyze surface information based on the appearance of contour lines or other patterned illumination.

Structured light imaging has been used effectively for surface contour imaging of solid, highly opaque objects and has been used for imaging the surface contours for some portions of the human body and for obtaining detailed data about skin structure. Structured light imaging methods have also been applied to the problem of dental imaging, helping to provide detailed surface information about teeth and other intraoral features. Intraoral structured light imaging is now becoming a valuable tool for the dental practitioner, who can obtain this information by scanning the patient's teeth using an inexpensive, compact intraoral scanner, such as the Model CS3500 Intraoral Scanner from Carestream Dental, Atlanta, Ga.

There is significant interest in providing intraoral camera and scanner devices capable of generating images in real time. The advent of less expensive video imaging devices and advancement of more efficient contour image processing algorithms now make it possible to acquire structured light images without the need to fix the scanner in position for individually imaging each tooth. With upcoming intraoral imaging systems, it can be possible to acquire contour image data by moving the scanner/camera head over the teeth, allowing the moving camera to acquire a large number of image views that can be algorithmically fitted together and used to for forming the contour image.

Contour imaging uses patterned or structured light to obtain surface contour information for structures of various types. In structured light projection imaging, a pattern of lines or other shapes is projected toward the surface of an object from a given direction. The projected pattern from the surface is then viewed from another direction as a contour image, taking advantage of triangulation in order to analyze surface information based on the appearance of contour lines. Phase shifting, in which the projected pattern is incrementally spatially shifted for obtaining images that provide additional measurements at the new locations, is typically applied as part of structured light projection imaging, used in order to complete the contour mapping of the surface and to increase overall resolution in the contour image.

In order to use the advanced imaging capabilities that video offers for contour imaging of dental features, a number of new problems must be addressed. One difficulty relates to the raw amount of data that is obtained in continuously scanning and collecting structured light images in video mode. Data continues to be acquired even where the scanner is moved slowly through the patient's mouth or if the scanner is placed on the dental work-table. Data redundancy can result, obtaining excessively large amounts of image data over the same area of the mouth or outside of the patient's mouth. Storage and processing of this data requires some processor resources as well as making significant demands on memory capability. The net result can be inefficiencies in image processing needed for matching image content to portions of the mouth and related problems.

Thus, it can be appreciated that there is a need for apparatus and methods that capture video structured light image data more efficiently and reduce excessive data acquisition and storage demands for intra-oral imaging applications.

### SUMMARY

It is an object of the present invention to advance the art of dental imaging for surface contour characterization. It is a feature of the present invention that it uses information from the scanning apparatus for determining the relative movement of the camera with respect to imaged teeth and can adapt the rate of contour image capture based on this movement detection.

Among advantages offered by the apparatus and method of the present invention are automated image capture for contour imaging without added camera components and improved imaging of tooth surfaces.

These objects are given only by way of illustrative example, and such objects may be exemplary of one or more embodiments of the invention. Other desirable objectives and advantages inherently achieved by the disclosed methods may occur or become apparent to those skilled in the art. The invention is defined by the appended claims.

According to one aspect of the disclosure, there is provided a method for obtaining one or more 3D surface images of a tooth, the method executed at least in part by a computer repeats a sequence of acquiring a succession of images of the tooth from a scanner at a scanner acquisition rate and changing the scanner acquisition rate according to differences between successive images in the acquired succession of images.

### BRIEF DESCRIPTION OF THE DRAWINGS

The foregoing and other objects, features, and advantages of the invention will be apparent from the following more particular description of the embodiments of the invention, as illustrated in the accompanying drawings.

The elements of the drawings are not necessarily to scale relative to each other. Some exaggeration may be necessary in order to emphasize basic structural relationships or principles of operation. Some conventional components that would be needed for implementation of the described embodiments, such as support components used for providing power, for packaging, and for mounting and protecting system optics, for example, are not shown in the drawings in order to simplify description.

FIG. 1 is a schematic diagram that shows components of an imaging apparatus for surface contour imaging of a patient's teeth and related structures.

FIG. 2 shows schematically how patterned light is used for obtaining surface contour information using a handheld camera or other portable imaging device.

FIG. 3 shows an example of surface imaging using a pattern with multiple lines of light.

FIG. 4 is a schematic diagram that relates scanner acquisition rate to scanner movement under different conditions.

FIG. 5 is a logic flow diagram that shows a sequence for adjusting the scanner acquisition rate during a scanning operation.

### DETAILED DESCRIPTION OF THE EMBODIMENTS

The following is a detailed description of the preferred embodiments, reference being made to the drawings in which the same reference numerals identify the same elements of structure in each of the several figures.

Where they are used in the context of the present disclosure, the terms “first”, “second”, and so on, do not necessarily denote any ordinal, sequential, or priority relation, but are simply used to more clearly distinguish one step, element, or set of elements from another, unless specified otherwise.

As used herein, the term “energizable” relates to a device or set of components that perform an indicated function upon receiving power and, optionally, upon receiving an enabling signal.

Two lines of light, portions of a line of light, or other features in a pattern of structured illumination can be considered to be substantially “dimensionally uniform” when their line width is the same over the length of the line to within no more than +/−15 percent. As is described in more detail subsequently, dimensional uniformity of the pattern of structured illumination is used to maintain a uniform spatial frequency.

In the context of the present disclosure, the term “optics” is used generally to refer to lenses and other refractive, diffractive, and reflective components used for shaping and orienting a light beam.

In the context of the present disclosure, the terms “viewer”, “operator”, and “user” are considered to be equivalent and refer to the viewing practitioner, technician, or other person who may operate a camera or scanner and may also view and manipulate an image, such as a dental image, on a display monitor. An “operator instruction” or “viewer instruction” is obtained from explicit commands entered by the viewer, such as by clicking a button on the camera or by using a computer mouse or by touch screen or keyboard entry.

In the context of the present disclosure, the phrase “in signal communication” indicates that two or more devices and/or components are capable of communicating with each other via signals that travel over some type of signal path. Signal communication may be wired or wireless. The signals may be communication, power, data, or energy signals. The signal paths may include physical, electrical, magnetic, electromagnetic, optical, wired, and/or wireless connections between the first device and/or component and second device and/or component. The signal paths may also include additional devices and/or components between the first device and/or component and second device and/or component.

In the context of the present disclosure, the terms “camera” and “scanner” are used interchangeably, as the description relates to structured light images successively projected and captured by a camera device operating in a continuous acquisition or video mode.

In the context of the present disclosure, the phrase “3D surface imaging” refers to any of a number of techniques that are used to obtain 3D surface, contour, and depth information for characterizing the surface features of a subject. “Range imaging” is one class of 3D surface imaging that uses image content acquired from 2D image sensors. There are a number of types of 3D surface imaging approaches, each using information from a sequence of 2D images. 3D surface imaging techniques for acquiring 3D surface images familiar to those skilled in the imaging arts and suitable for use in various embodiments of the present disclosure include the following:
(i) Contour imaging using structured light illumination, as described in more detail subsequently.
(ii) Depth from focus imaging. Also termed “structure from focus”, methods applying this technique sweep the object plane using an optical scanner moving toward or away from the subject along a depth direction. This moving element allows the acquisition of a stack of 2D images, each acquired image corresponding to an observation at a specific depth (like a microscope). Each path of light from the scanner intersects the images from the volumes at various depths. An algorithm processes the acquired image data and determines a best depth value by analyzing local blur information, related to spatial frequencies. This gives a set of 3D points in focus which is representative of features on the observed surface.
(iii) Structure from motion. Algorithms that use structure from motion (SFM) provide a type of range imaging that allows depth estimation to be obtained from a sequence of 2D images from a camera that is moving about a 3D structure. Edge features and other salient features are tracked from image to image and used to characterize the surface contour as well as camera motion.
(iv) Active/passive stereophotogrammetry or single camera photogrammetry (also called SLAM (Simultaneous Localization And Mapping): Similar to SFM, two images of the same object are taken under slightly different observation orientations (either using two cameras fixed relative spatial position or a single camera moved between two positions). Similar features (or landmarks) are paired between those two images. The set of corresponding landmarks is used to estimate the observation orientations (the relative camera orientation if not already known) and also determine a set of 3D points.
(v) Optical coherent tomography (OCT)/ultrasound. Both of these methods are echo-location techniques using either light or sound waves. OCT uses correlation with a reference pulse. A variable delay is equivalent to scanning in the depth direction. Ultrasound electronics can directly record a returned signal from the transducer which is converted to depth information. An array of sources or a spatial or an angular sweeping technique may be used to collect depth information along different paths. The echo from each path locates a 3D surface point. The combination of numerous 3D surface points defines a 3D surface contour.
(vi) Time of flight (TOF). TOF methods measure the propagating time of reflected light to extract depth information from an object. One type of TOF uses a pulse signal and a synchronized camera to record the flight time. The depth can be calculated using constant light speed. Another type of TOF uses a modulating wave and a synchronized camera to record the phase shifting. The depth can be estimated from the shifted phase and the light speed.
(vii) Structure from shading (SFS). Methods using the SFS approach reconstruct the 3D shape of a surface from a single image in which pixel intensity along a surface relates to the angle between the illumination source for the surface and the surface normal at that pixel.

In the context of the present disclosure, the terms “structured light illumination” or “patterned illumination” are used to describe the type of illumination that is used for structured light projection imaging or “contour” imaging that characterizes tooth shape. The structured light pattern itself can include, as patterned light features, one or more lines, circles, curves, or other geometric shapes that are distributed over the area that is illuminated and that have a predetermined spatial and temporal frequency. One exemplary type of structured light pattern that is widely used for contour imaging is a pattern of evenly spaced lines of light projected onto the surface of interest.

In the context of the present disclosure, the term “structured light image” refers to the image that is captured during projection of the light pattern or “fringe pattern” that is used for characterizing the tooth contour. “Contour image” and “contour image data” refer to the processed image data that are generated and updated from structured light images.

As was noted earlier in the background section, images that are used to characterize surface structure can be fairly large. The continuous acquisition of these images can be a significant burden for memory and storage circuitry that serves the imaging apparatus. For example, structured light images can be acquired with the scanner operating in video mode, so that structured light patterns are continuously directed to the tooth and images successively acquired. However, this can lead to significant data redundance and the need for a substantial amount of processing of duplicate data or image content that has no value for contour imaging of the mouth, such as data obtained when the camera is momentarily placed on the dental worktable or other work surface. On/off switches or manual controls for adjusting scanner acquisition rate can prove cumbersome in practice. Embodiments of the present disclosure address this problem by adjusting the image acquisition rate based on feedback obtained from monitoring the image processing algorithms or other source indicative of scanner use and activity.

For the sake of illustration, the description that follows relates to a 3D surface imaging embodiment that employs contour imaging using structured light illumination. As noted previously, other types of 3D surface imaging can alternately be used for embodiments of the present disclosure.

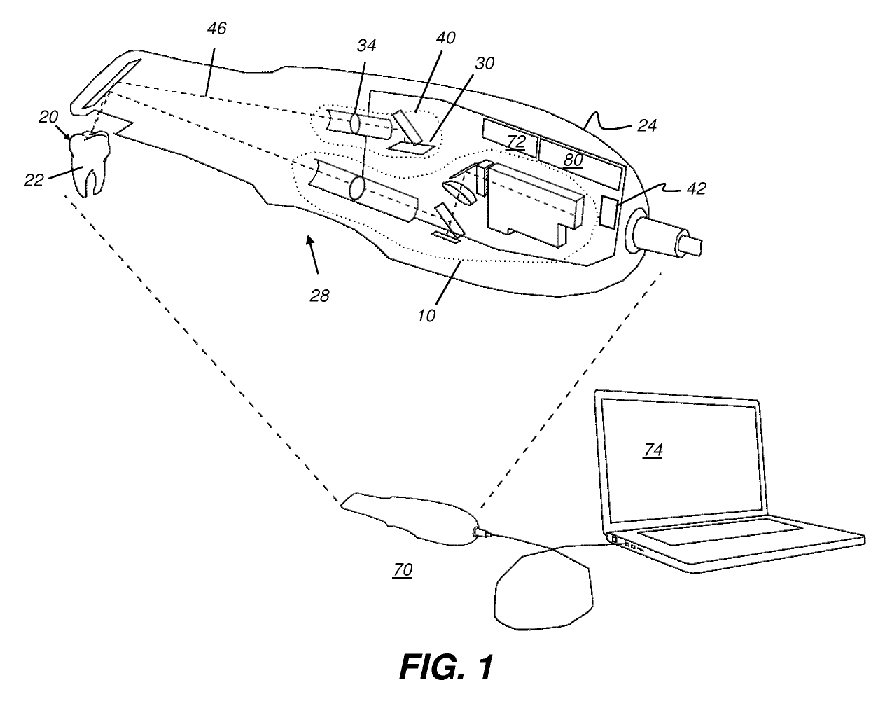

FIG. 1 is a schematic diagram showing an imaging apparatus 70 that operates as a video camera 24 for image capture as well as a scanner 28 for projecting and imaging to characterize surface contour using structured light patterns 46. A handheld imaging apparatus 70 uses a video camera 24 for image acquisition for both contour scanning and image capture functions according to an embodiment of the present disclosure. A control logic processor 80, or other type of computer that may be part of camera 24, controls the operation of an illumination array 10 that generates the structured light and directs the light toward a surface position and controls operation of an imaging sensor array 30. Image data from surface 20, such as from a tooth 22, is obtained from imaging sensor array 30 and stored as video image data in a memory 72. Imaging sensor array 30 is part of a sensing apparatus 40 that includes an objective lens 34 and associated elements for acquiring video image content. Control logic processor 80, in signal communication with camera 24 components that acquire the image, processes the received image data and stores the mapping in memory 72. The resulting image from memory 72 is then optionally rendered and displayed on a display 74 that can be part of a computer 75. Memory 72 may also include a display buffer. One or more sensors 42, such as a motion sensor, can also be provided as part of scanner 28 circuitry.

In structured light imaging, a pattern of lines or other shapes is projected from illumination array 10 toward the surface of an object from a given angle. The projected pattern from the illuminated surface position is then viewed from another angle as a contour image, taking advantage of triangulation in order to analyze surface information based on the appearance of contour lines. Phase shifting, in which the projected pattern is incrementally shifted spatially for obtaining additional measurements at the new locations, is typically applied as part of structured light imaging, used in order to complete the contour mapping of the surface and to increase overall resolution in the contour image.

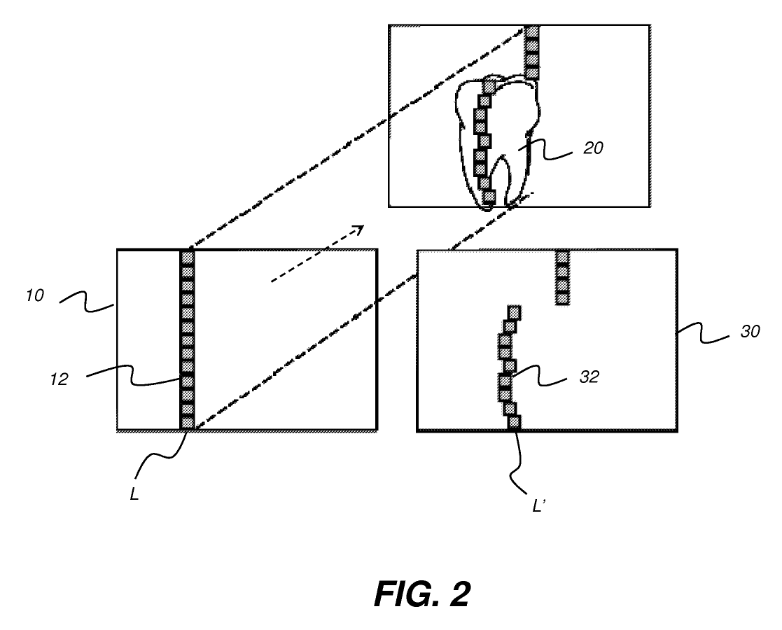

The schematic diagram of FIG. 2 shows, with the example of a single line of light L, how patterned light is used for obtaining surface contour information by a scanner using a handheld camera or other portable imaging device. A mapping is obtained as an illumination array 10 directs a pattern of light onto a surface 20 and a corresponding image of a line L′ is formed on an imaging sensor array 30. Each pixel 32 on imaging sensor array 30 maps to a corresponding pixel 12 on illumination array 10 according to modulation by surface 20. Shifts in pixel position, as represented in FIG. 2, yield useful information about the contour of surface 20. It can be appreciated that the basic pattern shown in FIG. 2 can be implemented in a number of ways, using a variety of illumination sources and sequences for light pattern generation and using one or more different types of sensor arrays 30. Illumination array 10 can utilize any of a number of types of arrays used for light modulation, such as a liquid crystal array or digital micromirror array, such as that provided using the Digital Light Processor or DLP device from Texas Instruments, Dallas, Tex. This type of spatial light modulator is used in the illumination path to change the light pattern as needed for the mapping sequence.

By projecting and capturing images that show structured light patterns that duplicate the arrangement shown in FIG. 1 multiple times, the image of the contour line on the camera simultaneously locates a number of surface points of the imaged object. This speeds the process of gathering many sample points, while the plane of light (and usually also the receiving camera) is laterally moved in order to “paint” some or all of the exterior surface of the object with the plane of light.

A synchronous succession of multiple structured light patterns can be projected and analyzed together for a number of reasons, including to increase the density of lines for additional reconstructed points and to detect and/or correct incompatible line sequences. Use of multiple structured light patterns is described in commonly assigned U.S. Patent Application Publications No. US2013/0120532 and No. US2013/0120533, both entitled “3D INTRAORAL MEASUREMENTS USING OPTICAL MULTILINE METHOD” and incorporated herein in their entirety.

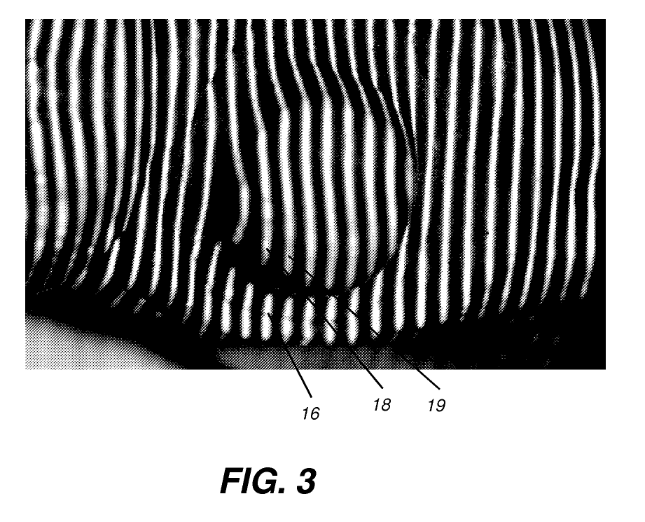

FIG. 3 shows surface imaging using a pattern with multiple lines of light. Incremental shifting of the line pattern and other techniques help to compensate for inaccuracies and confusion that can result from abrupt transitions along the surface, whereby it can be difficult to positively identify the segments that correspond to each projected line. In FIG. 3, for example, it can be difficult over portions of the surface to determine whether line segment 16 is from the same line of illumination as line segment 18 or adjacent line segment 19.

By knowing the instantaneous position of the camera and the instantaneous position of the line of light within an object-relative coordinate system when the image was acquired, a computer and software can use triangulation methods to compute the coordinates of numerous illuminated surface points. As the plane is moved to intersect eventually with some or all of the surface of the object, the coordinates of an increasing number of points are accumulated. As a result of this image acquisition, a point cloud of vertex points or vertices can be identified and used to represent the extent of a surface within a volume. The points in the point cloud then represent actual, measured points on the three dimensional surface of an object.

Conventional structured light contour imaging fixes the scanner or camera at a fixed point relative to the subject, then projects a series of structured light patterns and acquires the corresponding images with the camera at its fixed position. Although there can be some fluctuation in the scanner acquisition rate due to factors such as processing or transmission protocol speeds, scanner acquisition time is generally fixed, so that each successive image is acquired within a predetermined time period. In general, establishment of a fixed geometric point of reference is common to conventional structured light imaging techniques, as is the use of a scanning rate and sequence that does not vary with scanning conditions.

Methods of the present disclosure that use video contour imaging change the scanning paradigm and adapt the scanner acquisition rate according to the relative movement of the scanner during image acquisition. Thus, embodiments of the present disclosure help to adapt scanner behavior so that it is more suitable for handheld use in intra-oral scanning applications.

Video scanning allows changes in the relative position of the scanner to the scanned subject during acquisition of the structured light images by employing various types of matching algorithms that provide sufficient data for matching detected features of the subject as the camera is moved. Matching algorithms can enable point clouds reconstructed from scanning to be registered to each other, using techniques based on distance and weighted or cost functions familiar to those skilled in the 3-D imaging arts. Matching algorithms use techniques such as view angle computation between features and polygon approximations for mesh arrangement of the point cloud, alignment of centers of gravity or mass, and successive operations of coarse and fine alignment matching to register and adjust for angular differences between existing and newly generated point clouds. Registration operations for spatially correlating point clouds can include rotation, scaling, translation, and similar spatial operations that are familiar to those skilled in the imaging arts for use in 3-D image space.

Some types of matching algorithms work with an existing assembled structure, checking the relative position of newly scanned image content to previously scanned image content that has been processed and used to form the structure. Thus, for example, for intra-oral scanning, each newly acquired scan can be checked to determine if it includes tooth features that have been identified from preceding scan content. When such features can be located and matched with the newly scanned content, successful processing of the scanned content can proceed.

As is noted in the background material given previously, continuous scanning can generate a significant amount of image content, not all of which is useful for characterizing the 3-D surface contour. Some of the scanned images may be irrelevant, such as images acquired while the scanner is being moved into position or images acquired while the scanner is docked or at rest. Other image content may be redundant, such as where the same portion of the subject, such as the same tooth in intra-oral imaging, is continuously scanned. Embodiments of the present disclosure address this problem by automatically adjusting the scan rate according to feedback from the detected scan content. This automatic adjustment can accelerate the image acquisition rate of the scanner when the scanner is moved quickly over the subject of interest or slow the image acquisition rate appropriately when the scanner is moved more slowly over a region, even scanning at a very slow rate when the scanner is stationary or docked on the dental table or other holding surface.

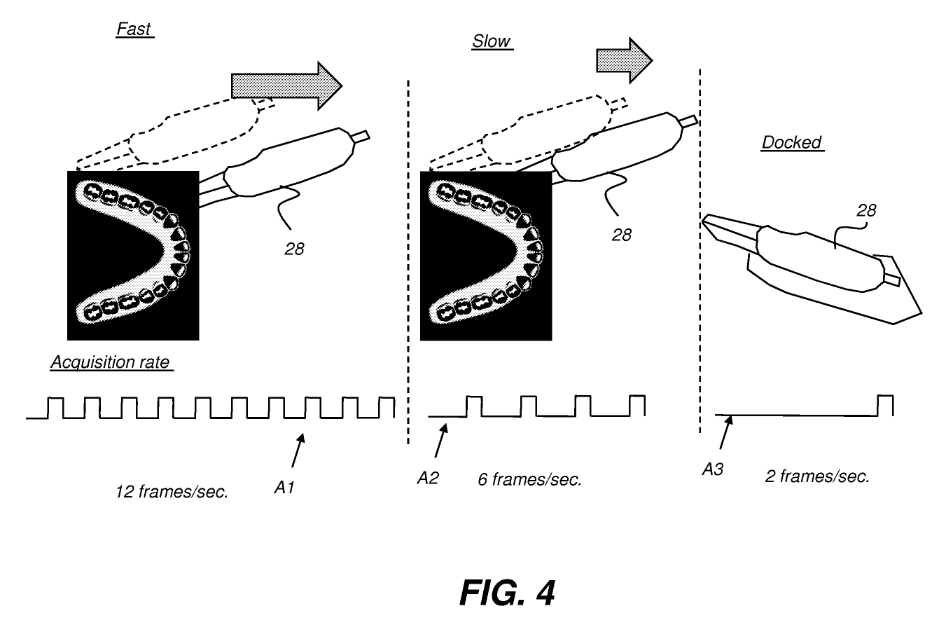

By way of example, the schematic diagram of FIG. 4 shows different scanning acquisition rates that can be used for a handheld intraoral scanner 28, such as that provided by imaging apparatus 70 in FIG. 1. Exemplary conditions for different scanner 28 speeds are shown from left to right in FIG. 4. At furthest left, scanner 28 is moved quickly along the side of a dental arch 110, as indicated by the arrow and phantom outline. Detection of this condition causes scanner 28 to maintain a relatively high acquisition rate A1, shown in this example as 12 acquisitions/sec. Moving to the right in FIG. 4, slowed movement of scanner 28, as indicated by the shortened arrow and phantom outline, causes the frame acquisition rate A2 to reduce by half, to 6 acquisitions/sec. When scanner 28 is docked or otherwise stationary and outside the mouth, or simply without the intended subject within its field of view (FOV), acquisition rate A3 is used, obtaining only a few acquisitions/sec for analysis until the subject returns to the scanner field of view.

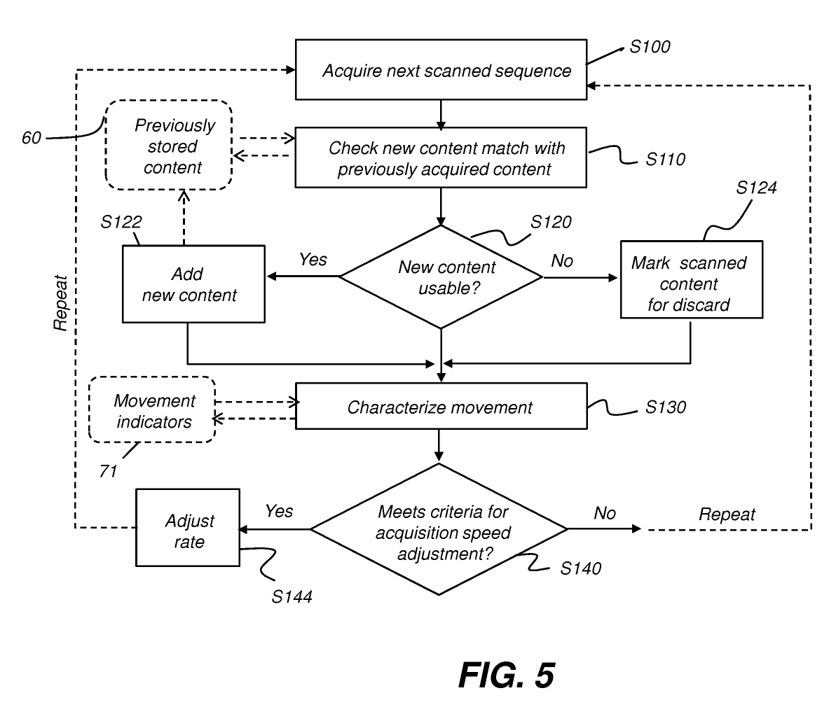

The logic flow diagram of FIG. 5 shows a sequence for monitoring and adjusting the scanner acquisition rate according to an embodiment of the present disclosure. In an image acquisition step S100, the scanner captures a sequence of images, which may consist of a single image or, alternately, may be a series of images such as images obtained from projecting a set of incrementally shifted lines or patterns onto the subject. A content evaluation step S110 then checks the newly acquired image content against previously stored content 60, including processed content that has been used to generate contour structure data. A decision step S120 determines whether or not the newly acquired image content is usable, based on results from content evaluation step S110. If the content is suitable for storing and processing along with existing content, an add content step S122 executes, adding the newly acquired scan content to existing image content and continuing. Otherwise, the newly acquired scan content is marked for discard in a discard content step S124. A movement characterization step S130 then checks the amount of relative movement of the scanner to the subject, based on the added image content or on the content that is to be discarded, and, optionally, on other movement indicators 71, as described in more detail subsequently. A decision step S140 determines whether or not criteria have been met for adjusting the image acquisition rate of the scanner. An adjustment step S144 adjusts the scanner acquisition rate to be faster or slower according to the detected movement and image content parameters. The processing repeats as long as the scanner is active, continuously acquiring and checking each new sequence of scanned image data to determine if the data includes useful content for surface contour characterization and whether or not scanner rate adjustment is needed. The surface contour results can be displayed, with the display continuously updated during image acquisition, for example.

It can be appreciated that the sequence shown in FIG. 5 can be performed as the scanner is being operated in normal use and may apply a number of approaches for assessing suitable scanner acquisition speed, based on image content as well as on other types of movement indicators.

Various types of movement indicators 71 can be used, individually or in combination, to provide a measurement of speed of movement of the scanner relative to a tooth. Movement indicators 71 can include a signal or signals obtained from physical motion sensors, including, but not limited to, a device such as an accelerometer, a gyroscope, or a magnetometer, for example. A 2D image sensor can provide an alternate type of movement sensing; the 2D image sensor can capture video images, structured pattern images, or shading images, for example. A 3D imaging scanner can also provide movement sensing data, obtaining 3D contour images and registration relations between different contour images. Movement indicators of various types can be used with the dental 3D scanner as well as with other imaging systems, such as a dental x-ray or CBCT systems.

For example, one type of movement indicator can be an accelerometer or other type of motion sensor 42 that is coupled to scanner 28 and is in signal communication with control logic processor 80, as described earlier with reference to FIG. 1. An accelerometer can be used in conjunction with image feedback movement indicators. According to an embodiment of the present disclosure, an accelerometer provides signals that are used to initiate or to terminate scanning, as well as to determine a suitable scan rate.

As described previously, matching algorithms can be used to help determine an appropriate scanner speed based on image content. Scanner acquisition rate can be based on the ongoing results from contour image processing. For each assembled 3D view, the relative position of the view to an overall construction of tooth structure can determine how well adjusted the scan rate is at a particular time. The standard assembly sequence for 3D construction can thus serve as a guide to whether or not an increase or decrease in scanner acquisition rate would be helpful.

Estimates of scanner movement speed and acceleration can be dynamically obtained by identifying scanned image content and features and calculating recommended or targeted spatial and periodic intervals between image captures. For example, it may be determined that the scanner movement between image captures should be no more than some number of millimeters or some fraction of a millimeter. Alternately, it may be determined that the angular change of the scanner relative to an identified feature should not exceed a certain number of minutes or degrees between images.

In addition to changing the scanner acquisition rate, embodiments of the present disclosure also provide the capability for modifying the projected scan pattern or other scanner behavior according to the image content that is obtained. For example, for a highly detailed surface, it may be useful for the scanner to use a projected pattern having narrower gaps between projected lines or to project the lines or other pattern elements with a different angular orientation that can be more advantageous with different surface contours.

In an embodiment of the present disclosure, the scanning acquisition rate is changed based on the scanner speed evaluation from 2D video images only. One advantage of this approach is that, even if 3D reconstruction is significantly slowed, the scanner can still acquire 2D video images displayed on the user interface.

Reference application “A METHOD AND SYSTEM FOR THREE-DIMENSIONAL IMAGING” PCT/CN2013/072424 describes how to obtain a 2D homography matrix H from two 2D video frames taken at times t₁ and t₂. For an affine homography H, the general 3×3 matrix representation can be written:

$$H = [ \begin{matrix}{h}_{11} & {h}_{12} & {l}_{x} \\ {h}_{21} & {h}_{22} & {l}_{y} \\ 0 & 0 & 1\end{matrix} ]$$

A simple criterion would be the estimate of 2D speed (termed S, following) obtained by computing the ratio of distance over the time difference:

$$S = \frac{\sqrt{{l}_{x}^{ 2} + {l}_{y}^{ 2}}}{{t}_{2} - {t}_{1}}$$

The obtained speed can correspond to a predetermined table of recommended acquisition rates, which is designed to have a predetermined overlap of reconstructed 3D range images for successful matching.

In an alternate embodiment of the present disclosure, the scanning acquisition rate is changed due to one or more consecutive structured light images. For example, since the contrast of the structured light pattern varies with the depth of the surface, the relationship between image contrast and depth can be roughly established. Then, if the depth of one or more images is considered to be within a valid range, the acquisition rate can be immediately increased to a high value. Otherwise the acquisition rate can be reduced.

According to an embodiment of the present disclosure, the scanning acquisition rate is changed when one or more consecutive 3D frames fail to match onto the 3D model that has already been reconstructed. This can indicate that there might not be any object in the scanner field of view or that the object that is being observed does not belong to the teeth surface being reconstructed.

In an alternate embodiment of the present disclosure, acquired 3D range images are used to estimate a range image success rate, but are not displayed to the user. If the range image success rate exceeds a predetermined threshold, the acquisition rate is immediately increased to a higher value; otherwise, the acquired 3D range images are discarded. This behavior can be useful when acquisition must not start too early to avoid acquisition of soft tissues, or acquisition while the scanner lies on the dental worktable. The predetermined threshold indicates sufficient confidence that the operator has reached a region of interest where acquisition should proceed at a faster rate.

According to an embodiment of the present disclosure, the scanning acquisition rate is related to an estimate of scanner speed from matching results. Let M₁ and M₂ be the positions and orientation of the scanner relative to an arbitrary coordinate system for the 3D model being reconstructed (usually referenced to the first 3D capture) for acquisition times t₁ and t₂. Here, M1 and M2 can be represented in a general way by two 4×4 matrices describing a rigid 3D transform, as follows:

$$M = [ \begin{matrix}{R}_{11} & {R}_{12} & {R}_{13} & {l}_{x} \\ {R}_{21} & {R}_{22} & {R}_{23} & {l}_{y} \\ {R}_{31} & {R}_{32} & {R}_{33} & {l}_{z} \\ 0 & 0 & 0 & 1\end{matrix} ]$$

Where Rᵢⱼ are the coefficients of a rotation matrix and (lₓ,l_{y},l_{z}) is a 3D translation vector. Various models of scanner displacement can be used. More basic, first-order models would assume constant speed (no acceleration). Second-order models also estimate scanner acceleration and require three different times and their associated scanner position matrices. Below, an example is given for first-order model. The scanner velocity can be estimated between those two times using a matrix power formula:

V₁₂=(M₂M₁⁻¹)^{1/(t}^{₂}^{-t}^{₁}⁾

Where M₁⁻¹ is the inverse of matrix M₁ and (M₂M₁⁻¹) is the displacement from position 1 to 2. The matrix exponent 1/(t₂-t₁) requires standard matrix algebra. In turn, if scanner speed was also available for earlier times (t₀ and t₁), the same formula can be used to derive the acceleration A₀₂ from the two speed estimations. The displacement model can then be used to predict the scanner location at a future time t₃:

\hat{V}₂₃=A₀₂^{(t}^{₃}^{-t}^{₂}⁾V₁₂

\hat{M}₂=\hat{V}₂₃^{(t}^{₃}^{-t}^{₂}⁾M₂

Where V₂₃ is the estimated scanner velocity using current velocity and acceleration and M₃ is the estimated scanner position.

Each possible scanner acquisition rate corresponds to a different future acquisition time t₃. The best acquisition rate can be computed from the estimated scanner displacement (M₃M₂⁻¹)=V₂₃. For instance, one could set a predefined target translation distance lₜₕᵣₑₛₕ and an angular threshold θₜₕᵣₑₛₕ and pick the slowest acquisition rate such that the translation doesn't exceed lₜₕᵣₑₛₕ and angular rotation doesn't exceed θₜₕᵣₑₛₕ.

According to an embodiment of the present disclosure, the acquisition rate may be decreased down to 0 frame/sec. This may happen if the scanner is believed to be almost still, or if multiple 3D views have consecutively failed matching, for example. Decreasing the scanning speed to 0 provides an automatic way to stop the capture sequence without having to press on a button or enter an operator command. This allows automatic start of other steps in the workflow.

According to an embodiment of the present disclosure, the acquisition rate may further be controlled by position sensors such as accelerometer, gyroscope, or magnetometer, for example. Those hardware components can detect if the scanner is moving relative to the earth's coordinate system or relative to the scanner's previous location. Detected movement indicates that the dentist is scanning or is about to scan. For instance, if the acceleration from the acceleration sensor value goes above a predetermined threshold, the acquisition rate may be increased to a positive value, which resumes the 3D capture sequence automatically. This is one possible method of resuming the scan once the scanner speed has been set to 0.

The surface contour image that is obtained using the apparatus and methods of the present disclosure can be displayed, processed, stored, transmitted, and used in a number of ways. Contour data can be displayed on display 74 (FIG. 1) and can be input into a system for processing and generating a restorative structure or can be used to verify the work of a lab technician or other fabricator of a dental appliance. This method can be used as part of a system or procedure that reduces or eliminates the need for obtaining impressions under some conditions, reducing the overall expense of dental care. Thus, the imaging performed using this method and apparatus can help to achieve superior fitting prosthetic devices that need little or no adjustment or fitting by the dentist. From another aspect, the apparatus and method of the present invention can be used for long-term tracking of tooth, support structure, and bite conditions, helping to diagnose and prevent more serious health problems. Overall, the data generated using this system can be used to help improve communication between patient and dentist and between the dentist, staff, and lab facilities.

Consistent with an embodiment of the present invention, a computer program utilizes stored instructions that perform on image data that is accessed from an electronic memory. As can be appreciated by those skilled in the image processing arts, a computer program for operating the imaging system in an embodiment of the present disclosure can be utilized by a suitable, general-purpose computer system, such as a personal computer or workstation. However, many other types of computer systems can be used to execute the computer program of the present invention, including an arrangement of networked processors, for example. The computer program for performing the method of the present invention may be stored in a computer readable storage medium. This medium may comprise, for example; magnetic storage media such as a magnetic disk such as a hard drive or removable device or magnetic tape; optical storage media such as an optical disc, optical tape, or machine readable optical encoding; solid state electronic storage devices such as random access memory (RAM), or read only memory (ROM); or any other physical device or medium employed to store a computer program. The computer program for performing the method of the present disclosure may also be stored on computer readable storage medium that is connected to the image processor by way of the internet or other network or communication medium. Those skilled in the art will further readily recognize that the equivalent of such a computer program product may also be constructed in hardware.

It should be noted that the term “memory”, equivalent to “computer-accessible memory” in the context of the present disclosure, can refer to any type of temporary or more enduring data storage workspace used for storing and operating upon image data and accessible to a computer system, including a database, for example. The memory could be non-volatile, using, for example, a long-term storage medium such as magnetic or optical storage. Alternately, the memory could be of a more volatile nature, using an electronic circuit, such as random-access memory (RAM) that is used as a temporary buffer or workspace by a microprocessor or other control logic processor device. Display data, for example, is typically stored in a temporary storage buffer that is directly associated with a display device and is periodically refreshed as needed in order to provide displayed data. This temporary storage buffer is also considered to be a type of memory, as the term is used in the present disclosure. Memory is also used as the data workspace for executing and storing intermediate and final results of calculations and other processing. Computer-accessible memory can be volatile, non-volatile, or a hybrid combination of volatile and non-volatile types.

It will be understood that the computer program product of the present disclosure may make use of various image manipulation algorithms and processes that are well known. It will be further understood that the computer program product embodiment of the present disclosure may embody algorithms and processes not specifically shown or described herein that are useful for implementation. Such algorithms and processes may include conventional utilities that are within the ordinary skill of the image processing arts. Additional aspects of such algorithms and systems, and hardware and/or software for producing and otherwise processing the images or co-operating with the computer program product of the present disclosure, are not specifically shown or described herein and may be selected from such algorithms, systems, hardware, components and elements known in the art.

While the invention has been illustrated with respect to one or more implementations, alterations and/or modifications can be made to the illustrated examples without departing from the spirit and scope of the appended claims. In addition, while a particular feature of the invention can have been disclosed with respect to one of several implementations, such feature can be combined with one or more other features of the other implementations as can be desired and advantageous for any given or particular function. The term “at least one of” is used to mean one or more of the listed items can be selected. The term “about” indicates that the value listed can be somewhat altered, as long as the alteration does not result in nonconformance of the process or structure to the illustrated embodiment. Finally, “exemplary” indicates the description is used as an example, rather than implying that it is an ideal. The presently disclosed embodiments are therefore considered in all respects to be illustrative and not restrictive. The scope of the invention is indicated by the appended claims, and all changes that come within the meaning and range of equivalents thereof are intended to be embraced therein.

1. A method for obtaining one or more 3D surface images of a tooth, the method executed at least in part by a computer and comprising a repeated sequence of:
acquiring a succession of images of the tooth from a scanner at a scanner acquisition rate; and
changing the scanner acquisition rate according to differences between successive images in the acquired succession of images.

2. A method for obtaining one or more 3D surface images of a tooth, the method executed at least in part by a computer and comprising a repeated sequence of:
acquiring a succession of images of the tooth from a scanner at a scanner acquisition rate;
measuring a speed of movement of the scanner relative to the tooth;
changing the scanner acquisition rate to acquire images at a slower or faster rate according to the determined relative speed of movement of the scanner;
and
rendering the one or more 3D surface images of the tooth to a display.

3. The method of claim 2 wherein measuring the relative speed of movement comprises comparing image content of two or more of the acquired images in the sequence.

4. The method of claim 2 wherein measuring speed of movement uses a structure from focus or defocus detection.

5. The method of claim 2 wherein measuring speed of movement uses a structure from motion detection.

6. The method of claim 2 wherein measuring speed of movement uses active photogrammetry.

7. The method of claim 2 wherein measuring speed of movement uses optical coherent tomography.

8. The method of claim 2 wherein determining the relative speed of movement of the scanner further comprises obtaining a signal from a sensor that is part of the scanner.

9. The method of claim 2 wherein acquiring the succession of images comprises projecting a periodic sequence of structured light patterns toward the tooth.

10. A method for obtaining a contour image of a tooth, the method executed at least in part by a computer and comprising a repeated sequence of:
projecting a periodic sequence of structured light patterns from a scanner toward the tooth at a scanning frequency;
acquiring a corresponding sequence of images of the projected structured light patterns at the scanning frequency and forming contour image data therefrom;
determining the relative speed of movement of the scanner according to the acquired image content for sequentially acquired images;
and
changing the periodic sequence of the scanning frequency to project and acquire images at a slower or faster rate according to the determined relative speed of movement of the scanner.

11. The method of claim 10 wherein projecting the sequence of structured light patterns comprises spatially shifting the structured light pattern.

12. The method of claim 10 further comprising displaying contour images during image acquisition by the scanner.

13. The method of claim 10 wherein the projected pattern changes according to variations in the surface contour.

14. An apparatus for imaging a tooth comprising:
an intra-oral scanner that has:
(i) an illumination source that projects a structured light pattern toward the tooth in response to a periodic excitation signal at a scanning frequency;
(ii) a detector that acquires successive structured light pattern images of the tooth at the scanning frequency;
(iii) a control logic processor programmed with instructions to process the acquired images from the detector, to determine the relative speed of movement of the scanner according to the acquired image content for sequentially acquired images, to increase or decrease the scanning frequency according to the determined relative speed, and to generate the excitation signal;
a computer that is in signal communication with the intra-oral scanner for receiving structured light image data acquired by the detector and for generating a contour image of the tooth;
and
a display that is in signal communication with the computer for display of contour images.
